(19) United States
(12) Plant Patent Application Publication
(10) Pub. No.: US 2003/0014786 P1
(43) Pub. Date: Jan. 16, 2003

(54) Hybrid tea rose plant named 'Meifebink'

(76) Inventor: Alain A. Meilland, Antibes (FR)

Correspondence Address: BURNS, DOANE, SWECKER & MATHIS, L.L.P., Post Office Box 1404, Alexandria, VA, 22313-1404, US

(21) Appl. No.: 09/880,914

(22) Filed: Jun. 15, 2001

(51) Int. Cl. A01H 5/00

(52) U.S. Cl. PLT/137

(57) ABSTRACT

A new and distinct variety of Hybrid Tea rose plant is provided that resulted as a mutation of the ‘Febesa’ variety (U.S. Plant Pat. No. 11,529). The new variety abundantly forms attractive double light red blossoms unlike the light Orient Pink blossoms of the ‘Febesa’ variety. Also, the blossoms of the new variety possess more petals than those of the ‘Febesa’ variety. The plant exhibits an erect strong vigorous growth habit, and dense decorative semi-glossy dark green foliage. Excellent disease resistance is displayed with respect to Powdery Mildew and Black Spot. The attractive dark green foliage contrasts nicely with the light red blossoms. The new variety is particularly well suited for cut flower production under commercial greenhouse growing conditions.

### SUMMARY OF THE INVENTION

The new Hybrid Tea variety of Rosa hybrida of the present invention was discovered during 1996 at Le Cannet des Maures, Var, France while growing among plants of the ‘Febesa’ variety (U.S. Plant Pat. No. 11,529). The new variety is believed to be the result of a natural mutation of unknown causation. The new variety was selected and was carefully preserved primarily because of its distinctive blossom coloration. Had this mutation not been discovered and preserved it would have been lost to mankind.

It was found that the new variety of the present invention:

(a) exhibits attractive large buds on straight erect stems,

(b) forms in abundance attractive light red blossoms having more petals than the ‘Febesa’ variety (U.S. Plant Pat. No. 11,529),

(c) forms attractive dark green semi-glossy foliage,

(d) exhibits excellent disease resistance with respect to Powdery Mildew and Black Spot, and

(e) is particularly well suited for cut flower production in the greenhouse.

The new variety well meets the needs of the horticultural industry and can be used to advantage for forming distinctive cut flowers in greenhouses under commercial growing conditions.

The new variety of the present invention can be readily distinguished from the parent ‘Febesa’ variety in view of the different flower coloration and a larger number of petals per flower. For instance, the ‘Febesa’ variety forms light Orient Pink flowers while those of the new variety of the present inventions are a deeper light red. The flowers of the ‘Febesa’ variety commonly possess approximately 23 petals on average while those of the new variety commonly possess approximately 38 petals on average.

The new variety has been found to undergo asexual propagation in France by a number of routes, including budding, grafting, and cuttage. Asexual propagation by the above-mentioned techniques at Le Cannet des Maures, Var, France has shown that the characteristics of the new variety are stable and are strictly transmissible by such asexual propagation from one generation to another.

The new variety has been named the ‘Meifebink’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age and were observed during January while budded on Rosa indica understock and growing in greenhouses at Le Cannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface;

FIG. 15—illustrates a specimen of a leaf with five leaflets—plan view—under surface; and

FIG. 16—illustrates a specimen of a leaf with seven leaflets—plan view—upper surface.

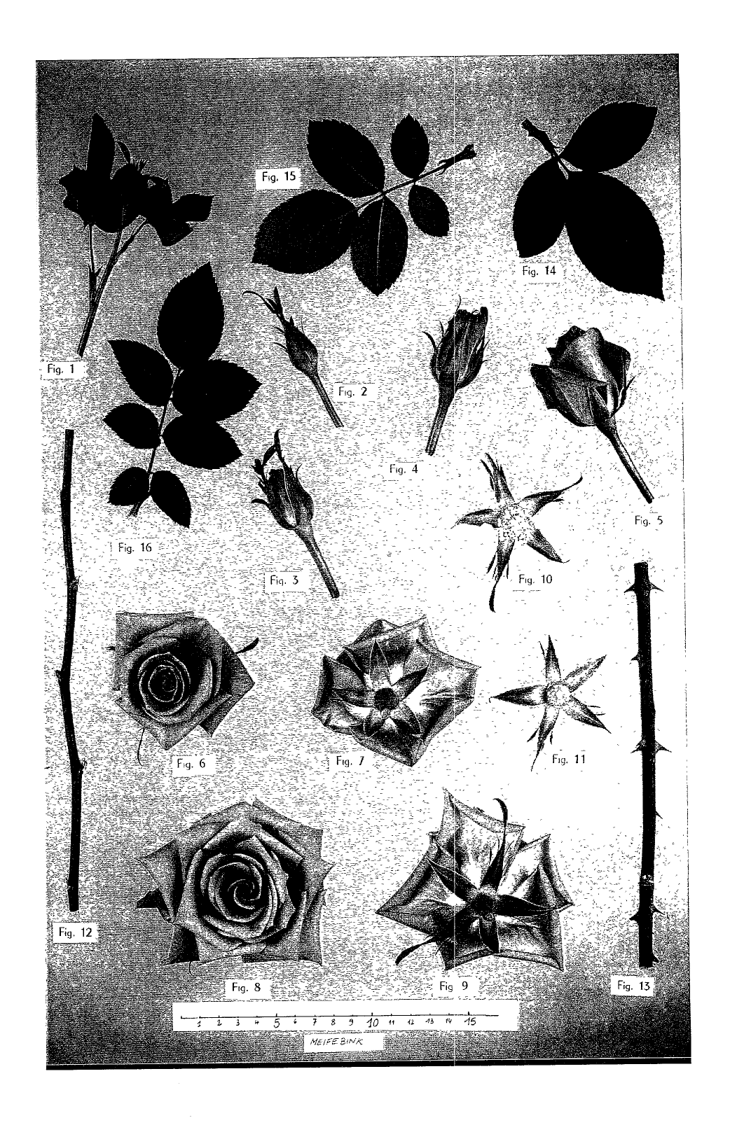

### DETAILED DESCRIPTION

The chart used in the identification of the colors is that of The Royal Horticultural Society (R.H.S. Colour Chart). The description is based on the observation of two year-old plants during April while budded on Rosa indica understock and growing in greenhouses at Le Cannet des Maures, Var, France.

Class: Hybrid Tea.

Plant:

height.—when pruned to a height of 85 cm, floral stems having, a length of approximately 50 to 80 cm commonly are produced at the end of the growing season.

| | |
|---|---|
| CLASS: | |
| Hybrid Tea. | |
| PLANT: | |
| height | when pruned to a height of 85 cm, floral |
| | stems having a length of approximately 50 |
| | to 80 cm commonly are produced at the end |
| | of the growing season. |
| habit | erect. |
| BRANCHES: | |
| color | young stems: Yellow-Green Group 146A. |
| | adult wood: Green Group 137A. |
| thorns | size: medium (as illustrated). |
| | quantity: fairly numerous (as illustrated). |
| | color: Greyed-Orange Group 167A. |
| LEAVES: | |
| stipules | adnate, pectinate, and narrow. |
| petioles | near Yellow-Green Group 147A to 147D and |
| | somewhat glandular. |
| leaflets | number: 3, 5, and 7 (most often). |
| | shape: elliptic. |
| | serration: regular (as illustrated). |
| | texture: somewhat consistent. |
| | general appearance: very dense, dark |
| | green, and semi-glossy. |
| | color (young foliage): |
| upper surface: | Yellow-Green Group 147A. |
| | under surface Yellow-Green Group 147B. |
| | color (adult foliage): |
| | upper surface: Green Group 139A. |
| | under surface: Green Group 138A. |
| INFLORESCENCE | |
| number | usually one per stem. |
| of flowers | |
| peduncle | near Green Group 138B and the length is |
| | approximately 8.5 cm on average. |
| sepals | near Green Group 137D in coloration, and |
| | commonly with some extensions (as |
| | illustrated). |
| buds | shape: elongated. |
| | size: large. |
| | length: approximately 5.5 cm on average. |
| flower | shape: commonly with a high center. |
| | diameter: approximately 9 cm on average. |
| | petal number: approximately 38 on average. |
| | This can be compared to approximately 20 |
| | to 24 for the ‘Febesa’ variety. |
| | color (when opening begins): |
| | upper surface: light red, near Red |
| | Group 48A. |
| | under surface: light red, near Red |
| | Group 48B. |
| | color (when blooming): |
| | upper surface: light red, near Red |
| | Group 48A. |
| | under surface: light red, near Red |
| | Group 48B. |
| | color (at end of opening): |
| | upper surface: light red, near Red |
| | Group 48A. |
| | under surface: light red, near Red |
| | Group 48B. |
| | fragrance: none. |
| | lasting quality: excellent, the blossoms |
| | commonly last approximately 15 days when |
| | cut and placed in a vase. |
| | petal shape: rounded with an obtuse |
| | base and reflexed edges with some quilling. |
| | stamen number: approximately 106 on |
| | average. |
| | anthers: ochre in coloration. |
| | filaments: yellowish in coloration. |
| | pistils: approximately 146 on average. |
| | stigmas: straw-colored. |
| | receptacle: smooth, light green in |
| | coloration, and funnel-shaped in |
| | longitudinal section. |
| DEVELOPMENT | |
| vegetation | very vigorous and strong. |
| blooming | very abundant. |
| resistance to | excellent especially with respect to Powdery |
| disease | Mildew and Black Spot. |

habit.—erect.

Branches:

color.—young stems: Yellow-Green Group 146A. adult wood: Green Group 137A.

thorns.—size: medium (as illustrated). quantity: fairly numerous (as illustrated). color: Greyed-Orange Group 167A.

Leaves:

stipules.—adnate, pectinate, and narrow.

petioles.—near Yellow-Green Group 147A to 147D and somewhat glandular.

leaflets.—number: 3, 5, and 7 (most often). shape: elliptic. serration: regular (as illustrated). texture: somewhat consistent. general appearance: very dense, dark green, and semi-glossy. color (young foliage): upper surface: Yellow-Green Group 147A. under surface: Yellow-Green Group 147B. color (adult foliage): upper surface: Green Group 139A. under surface: Green Group 138A.

Inflorescence

number of flowers.—usually one per stem.

peduncle.—near Green Group 138B and the length is approximately 8.5 cm on average.

sepals.—near Green Group 137D in coloration, and commonly with some extensions (as illustrated).

buds.—shape: elongated. size: large. length: approximately 5.5 cm on average.

flower.—shape: commonly with a high center.

diameter.—approximately 9 cm on average. petal number: approximately 38 on average. This can be compared to approximately 20 to 24 for the ‘Febesa’ variety. color (when opening begins): upper surface: light red, near Red Group 48A. under surface: light red, near Red Group 48B. color (whe n blooming: upper surface: light red, near Red Group 48A. under surface: light red, near Red Group 48B. color (at end of opening): upper surface: light red, near Red Group 48A. under surface: light red, near Red Group 48B. fragrance: none. lasting quality: excellent, the blossoms commonly last approximately 15 days when cut and placed in a vase. petal shape: rounded with an obtuse base and reflexed edges with some quilling. stamen number: approximately 106 on average. anthers: ochre in coloration. filaments: yellowish in coloration. pistils: approximately 146 on average. stigmas: straw-colored. receptacle: smooth, light green in coloration, and funnel-shaped in longitudinal section.

Development

vegetation.—very vigorous and strong.

blooming.—very abundant.

resistance to diseases.—excellent especially with respect to Powdery Mildew and Black Spot.

I claim:

1. A new and distinct variety of Hybrid Tea rose plant characterized by the following combination of characteristics:
(a) exhibits attractive large buds on straight erect stems,
(b) forms in abundance attractive light red blossoms having more petals than the ‘Febesa’ variety (U.S. Plant Pat. No. 11,529),
(c) forms attractive dark green semi-glossy foliage,
(d) exhibits excellent disease resistance with respect to Powdery Mildew and Black Spot, and
(e) is particularly well suited for cut flower production in the greenhouse; substantially as herein shown and described.
